(19) United States
(12) Patent Application Publication
(10) Pub. No.: US 2013/0101999 A1
(43) Pub. Date: Apr. 25, 2013

(54) MULTI-LEG LUMINESCENT NANOPARTICLES, MULTI-LEG LUMINESCENT NANOPARTICLE COMPOUNDS AND VARIOUS APPLICATIONS

(71) Applicants: Krishnan Chakravarthy, Williamsville, NY (US); Siddhartha Kamisetti, Cleveland, OH (US); Himanshu Subhash Amin, Solon, OH (US)

(72) Inventors: Krishnan Chakravarthy, Williamsville, NY (US); Siddhartha Kamisetti, Cleveland, OH (US); Himanshu Subhash Amin, Solon, OH (US)

(73) Assignee: NANOAXIS, LLC, Clarence, NY (US)

(21) Appl. No.: 13/626,823

(22) Filed: Sep. 25, 2012

Related U.S. Application Data
(60) Provisional application No. 61/538,961, filed on Sep. 26, 2011.

(51) Int. Cl. G01N 33/58

(52) U.S. Cl. CPC G01N 33/588; Y10S 977/813; Y10S 977/92; B82Y 15/00
USPC 435/6.11; 436/501; 435/7.94; 435/7.2; 435/6.12; 435/29; 435/7.23; 977/813; 977/920

(57) ABSTRACT

Multi-leg luminescent nanoparticles (“MLN's”) that can be paired to other MLN's as well s biological molecules to film branched multi-leg luminescent nanoparticles (“BMLN's) that can be used in biological multiplexing applications, imaging applications, biological detection applications and other biological applications.

### CROSS REFERENCED TO RELATED APPLICATION

This application claims the benefit of U.S. Provisional Patent Application Ser. No. 61/538,961 filed Sep. 26, 2011 and entitled “MLNs, MLN Compounds and Various Applications”, which is incorporated by reference herein in its entirety.

### TECHNICAL FIELD

This disclosure relates to a nanoparticle hetero-structure with one or more legs protruding from a base (herein referred to as a “multi-leg luminescent nanoparticle” or “MLN”) which can be made of one or more nanomaterials and is capable of binding to a multitude of particles, materials, genetic materials or materials with biological properties. The MLN is able to serve several therapeutic, diagnostic or imaging functions, such as the identification of numerous biological parameters for profiling several biological targets thus facilitating major advances in medical diagnostics, targeted therapeutics, molecular biology, cellular biology, patient care, and other life science applications.

### BACKGROUND

An understanding of cellular processes and structure is a very important aspect of biochemistry and medicine. Typically, fluorescence labeling is utilized to visualize a cellular target, whereby a fluorophore can bind to a target and imaged to recognize a molecule (e.g. antibody) associated with or attached to a the target to be identified. The current available fluorophore's, such as organic dyes, for diagnostic purposes are limited in its utility in that they lack photostability, brightness, are sensitive to pH, allow for a limited number of targets to be identified simultaneously, and other provide other such limitations. There is a need for diagnostic materials that provide high quantum yields, high molar extinction coefficients, broad absorption properties with narrow photoluminescence spectra's, have a variety of excitation wavelengths, are less susceptible to photobleaching, and allow for multiplexing applications.

Even with the advent of spherical quantum dots, there are limitations that need to be overcome with such quantum dots, including, but not limited to, the limitations in multiplexing applications, the limited number of unique spectral signatures, the inability to carry different recognition molecules on a single nanoparticle, the limitation that each spherical quantum dot only emits one single color, and other such limitations. A compound is needed that overcomes such problematic diagnostic, therapeutic, and imaging issues.

### SUMMARY

The following presents a simplified summary of the disclosure in order to provide a basic understanding of some aspects of the disclosure. This summary is not an extensive overview of the disclosure. It is intended to neither identify key or critical elements of the disclosure nor delineate any scope particular embodiments of the disclosure, or any scope of the claims. Its sole purpose is to present some concepts of the disclosure in a simplified form as a prelude to the more detailed description that is presented later.

In accordance with one or more embodiments and corresponding disclosure, various non-limiting aspects described in connection with MLN that exhibit. luminescent color characteristics. In accordance with a non-limiting embodiment, in an aspect, an article is provided comprising one or more legs extending from a base wherein the one or more leg and the base comprise a luminescent semiconductor nanoparticle; a shell that coats the one or more legs and the base: and a linking agent connected to the shell coated one or more legs and base, and configured to connect to an affinity molecule. In an aspect, the base and one or more legs are a II-VI semiconductor or a semiconductor.

The disclosure further discloses, to nanoparticle wherein the semiconductor is MgS, MgSe, MgTe, CaS, CaSe, CaTe, SrS, SrSe, SrTe, BaS, BaSe, BaTe, ZnS, ZnSe, ZnTe, CdS, CdSe, CdTe, HgS, HgSe, or HgTe. In another aspect, the multi-leg luminescent nanoparticle further comprises a glass coating on the shell. In yet another aspect, the linking agent can be any of a wherein the linking agent comprises any of a thiol moiety, F127COOH, alkyl group propyl group, N-(3-aminopropyl)-3-merecapto-benzamide, 3-aminopropyl-trimethoxysilane, 3-mercaptopropyltrimethoxysilane, 3-maleimidopropyl-trimethoxysilane, or 3-hydrazidopropyl-trimethoxysilane, diacetylenes, acrylates, acrylamides, dihydmlipoic acid, trioctyl phosphate, vinyl, and styryl.

In another aspect, further disclosed is a branched multi-leg luminescent nanoparticle compound, comprising: at least two bases, each of the at least two bases respectively having at least one leg protruding there from, wherein the at least two bases or the respective at least one legs are coupled, and wherein the at least two bases and the at least one lets comprise a luminescent semiconductor nanoparticle; a shell that coats the at least two bases and the at least one legs; and a linking agent connected to the shell coated the at least two bases and the at least one legs and configured to connect to an affinity molecule. In an aspect, the multi-leg luminescent nanoparticle can be synthesized by a microreactor process.

The following description and the annexed drawings set forth certain illustrative aspects of the disclosure. These aspects are indicative, however, of but a few of the various ways in which the principles of the disclosure may be employed. Other advantages and novel features of the disclosure will become apparent flow the following detailed description of the disclosure when considered in conjunction with the drawings.

### BRIEF DESCRIPTION OF THE FIGURES

FIG. 1 illustrates an example non-limiting MLN whereby the base length is indicated by “A”, the leg length is indicated by “B”, the leg width is indicated by “C” and the dotted lines indicate “n” legs.

FIG. 2A illustrates an example non-limiting a MLN with as core material (i.e. CdSe), a shell material surrounding the core material (ZnS), of which the core and shell material can be synthesized in a synthesis process occurring in a micro-reactor after which a linking agent is added F127COOH) then a biological material is conjugated to the multi-luminescent nanoparticle (i.e. antibody: FoxP3)

FIG. 2B illustrates an example non-limiting flow cytometer analysis of MLNs synthesized in a micro-reactor process with associated results.

FIG. 3 illustrates an example non-limiting diagram of the comparative advantage of multiplexing using a MLN (FIG. 3(2) and FIG. 3(3)) versus multiplexing using a spherical luminescent nanocrystal (FIG. 3(1)) as a function of different spectral signature outputs.

FIG. 4 illustrates an example non-limiting diagram of a MLN in Polymerase Chair Reaction technology applications.

FIG. 5 illustrates an example non-limiting diagram of a MLN in microarray technologies inc hiding but not limited to microarray analysis of DNA, RNA, or protein.

FIG. 6 illustrates an example non-limiting diagram of a MLN in a flow cytometer application.

FIG. 7 illustrates an example non-limiting diagram of a MLN in an in an in vitro application or in vivo application for cellular imaging or drug delivery analysis.

FIG. 8 illustrates an example non-limiting diagram of a MLN in various biological applications.

FIG. 9 illustrates an example non-limiting diagram of a MLN in a Western Blot application.

FIG. 10 illustrates a MLN in a Fluorescence Resonance Energy Transfer (FRET) analysis application.

FIG. 11 illustrates a MLN in a sandwich ELISA application.

### TERMS

The term “Base edge” is meant to describe the portion of the MLN where the legs extend.

The term “Base length” is meant to describe the distance from the centroid of the MLN to the base edge as indicated by “A” in the figure ranging anywhere from 0.001 nm to 999.999 nm in length.

The terms “bond” and “bonding” are meant to describe the adherence between the connecting molecule and the detectable substance. The adherence may comprise any sort of bond, including, but not limited to, covalent, ionic, or hydrogen bonding, Van der Waals' forces, magnetics, or mechanical bonding.

The term “Branched multi-leg luminescent nanoparticle compound” or “BMLN” is meant to describe one or more MLNs attached through bonding, magnetics, or conjugation.

The term “Leg” is meant to describe any protrusion from the base.

The term “Leg length” is meant to describe the distance from the centroid of the MLN to the tip end point of a leg indicated by “B” in the figure ranging anywhere from 0.001 nm to 999.999 nm in length.

The term “Leg width” is meant to describe the distance from the centroid of the MLN to the side point of a leg indicated by “C” in the figure ranging anywhere from 0.001 nm to 999.999 am in length.

The term “MLN” is meant to describe a multi leg luminescent nanoparticle.

The term “Spectral Signatures” is meant to describe the specific combination of reflected and absorbed electromagnetic. (EM) radiation at varying wavelengths which can uniquely identify an object

The term “Theranostic” is meant to describe a material that can both be imaged and can carry a therapeutic payload.

### DETAILED DESCRIPTION

### Overview

The advent of nanotechnology, introducing control over matter at the nanometer scale, has produced a class of materials known as a MLN (herein referred to as “MLN”) with novel properties, creating new possibilities in a diversity of domains. MLN's are versatile compounds that can be adjusted to take a variety of structures, forms, sizes, shapes and compositions (metal, polymer, semiconductor, dimensions of the particles which is a key factor leading to the possibility of enhancement and tailoring of many of the material properties, electrical, optical, chemical, biological, commercial materials, etc.) which, at such scale, become size and/or shape dependent. MLN's can possess enhanced mechanical properties, perform tunable light scattering or luminescence due to quantum size effects, and possess features, such as fine tunable spectral signatures that can be controlled by manipulating the six, shape, or number of legs, of the MLN.

An MLN is a nanomaterial comprised of one or more structured semiconductor nanomaterial that can absorb energy (such as electro magnetic radiation), can emit energy (such as electromagnetic radiation), scatter energy (such as electromagnetic radiation) when excited by an excitation source, diffract energy (such as electromagnetic radiation) when excited by an excitation source (such as a particle beam), and can demonstrate a detectable and measurable change in absorption and of emitting radiation in a narrow wavelength band or scattering of diffraction when excited. Only one common source for excitation of several MLN's need be used to excite the MLN's due to the broad bandwidth of the nanomaterials, that is, several MLN's can emit electromagnetic radiation at different frequencies after being excited by one single excitation source, thereby permitting multiplexing (simultaneous excitation of MLN's with different spectral signatures using a single common excitation source).

MLN's can be wed in a variety of detection, imaging, labeling, therapeutic and biological applications. These applications include, but are not limited to, polymerase chain reaction (PCR) technology applications, microarray technology applications, flow cytometer application, in vitro cellular imaging application, in vivo cellular imaging application, in vitro drug delivery application, in vivo drug delivery application, western blot application, FRET analysis application, sandwich ELISA application, genetic testing application, or other such biological applications. MLN's permit labeling of one or more biological materials (such as cellular parameters). Each MLN leg can be tuned in a different manner (by adding more legs, adjusting leg width, leg length, or adjusting, both leg length and leg width) to present a unique spectral signature fir each MLN thereby allowing numerous biological parameters. MLN's are versatile materials which present an opportunity to provide significant benefits and advancements to a variety of biological labeling, imaging, detection, and therapeutic applications.

### EXAMPLE EMBODIMENTS OF MLN'S, MLN COMPOUNDS AND VARIOUS MLN APPLICATIONS

The innovation is now described with reference to the drawings, wherein reference numerals are used to refer to like elements throughout. In the following description, for purposes of explanation, numerous specific details are set forth in order to provide a thorough understanding of this innovation. It may be evident, however, that the innovation can be practiced without these specific details. In other instances, well-known structures and components are shown in block diagram thrill in order to facilitate describing the innovation.

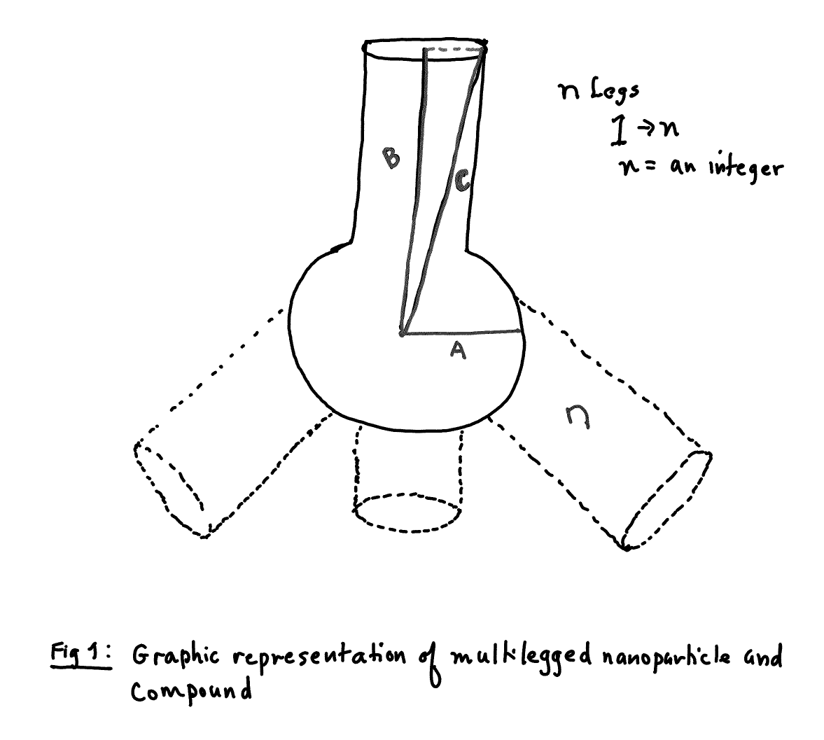

By way of introduction, the subject matter disclosed in this disclosure relates to nanoparticle articles known as MLN's which comprise one or more legs that exhibit luminescent color characteristics when excited by an excitation source. Referring now to the drawings, with reference initially to FIG. 1, MLN article 100 is shown. FIG. 1 shows a multi-legged luminescent nanoparticle, comprising: one or more legs extending from a base wherein the one or more legs and the base comprise a luminescent semiconductor nanoparticle a shell that coats the one or more legs and the base; and a linking agent connected to the shell coated one or more legs and base, and configured to connect to an affinity molecule.

A MLN comprises a base and one or more legs protruding from the base. In an aspect, a base describes the component of the MLN from which one or more legs extend. In an aspect, a one or more leg describes one or more protrusions from the base of the MLN. Each MLN has one or more legs extending from a base material whereby “n” in an integer. In an aspect, the addition of each new leg, gives a new spectral signature to the MLN that is different and identifiable from any MLN that lacks the same number of legs. For instance, and MLN whereby n=3 comprises three legs extending from the base, however an MLN whereby n=5 comprises five legs extending from the base. The MLN whereby n=3 has a unique spectral signature than the MLN whereby n=5. Furthermore, the unique spectral signature allows for each respective MLN to be detected separately due to such unique spectral signature.

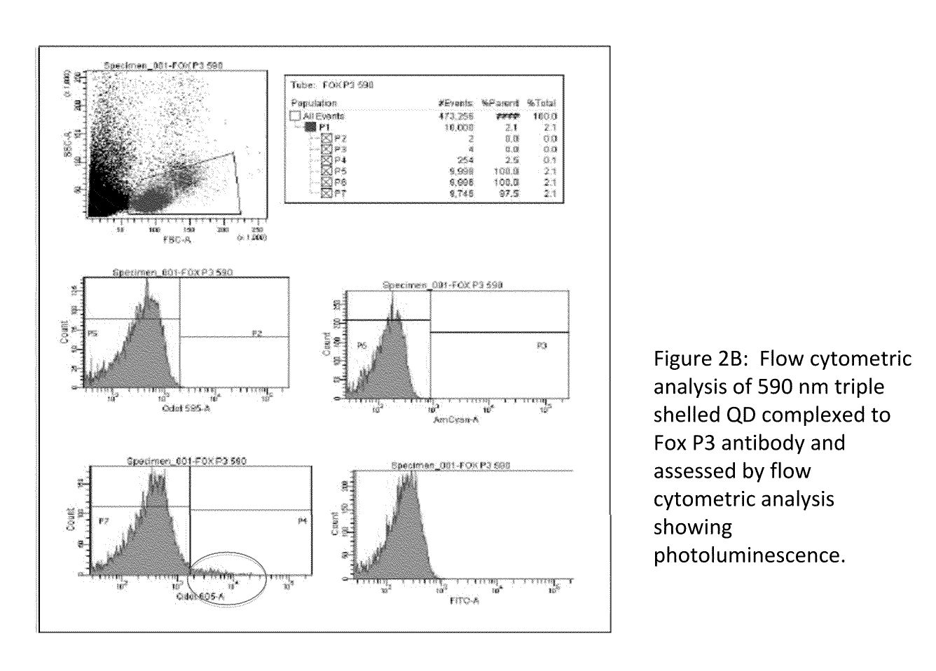

In an aspect, each leg comprises a leg length (indicated by “B” in FIG. 1 and FIG. 3) and a leg width, each of which can be respectively adjusted ibr each MLN. A leg length describes the distance from the centroid of the MLN to the tip end point of a leg indicated by “B” in FIG. 1 and FIG. 3 whereby B is a length greater than or equal to 0.001 nm and less than or equal to 999.999 nm. Each MLN leg has a leg width which is the distance from the centroid of the MLN to the side point of a leg indicated by “C” in the FIG. 1 and FIG. 3 whereby C is a length greater than or equal to 0.001 nm and less than or equal to 999.999 nm. Each leg length and each leg width can be adjusted to a distance greater than or equal to 0.001 nm and less than or equal to 999.99 nm and each leg on a particular MLN can be of different leg length or leg width.

Furthermore, in an aspect, an MLN has a base length (indicated by “A” in FIG. 1 and FIG. 3) which is the distance from the centroid of the MLN to the base edge as indicated by “A” in FIG. 1 and FIG. 3 wherein A is greater than or equal to 0.001 and less than or equal to 999.999 nm in distance and can be adjusted for each unique MLN. Additionally, in an aspect, an MLN comprises a base edge which is the point where each leg junctures with the base. Each leg can extend from a different base edge point thereby allowing for multiple base edges regions for each MLN.

In another aspect, each MLN may comprise legs of different leg lengths, legs of the same length OF a mixture of legs of the same length, legs of different leg length, legs of the same leg width's, legs of different leg, width's. MLN's of different base lengths, or MLN's of different base edge's. Each combination of leg length, leg width, base length or number of legs characterizing a respective MLN allows for the synthesis of numerous MLN's, each with a different spectral signature output thereby resulting in an detection of several uniquely identifiable MLN's. This simultaneous identification of several unique MLN's allows for multiplexing in biological applications whereby multiple biological parameters can be labeled and detected at the same time Furthermore, several MLN features (number of legs, leg length, leg width, base length, etc.) can be adjusted to allow for hundreds (and in some cases thousands) of unique MLN to respectively identify hundreds of unique biological parameters simultaneously.

In another aspect, MLN's can be grown, assembled or built into branched multi-leg luminescent nanoparticles (herein referred to as “BMLN's”), with each BMLN comprising a unique spectral signature, thereby further increasing the multiplexing capabilities of these versatile nanomaterial's. An MBN nanoparticle compound is comprised of two or more MLNs affixed through bonding, magnetics, and or complexation. In an aspect an MBN can either be affixed to or protruding from another multi-branched luminescent nanoparticle. Attachment of MBN's occur where any leg of a first MLN is attached to a base or leg of a second MLN through bonding, complexation or magnetics. In general, the stability of an MLN and of MBN's comprising one or more MLN's bound together are maintained by shelling the base and one or more legs of the MLN.

In an aspect the base and one or more legs are any one or more of a II-VI semiconductor material or a III-V semiconductor material. In another aspect, the base and one or more legs are made of a semiconductor wherein the semiconductor is one of: MgS, MgSe, MgTe, CaS, CaSe, CaTe, SrS, SrSe, SrTe, BaS, BaSe, BaTe, ZnS, ZnSe, ZnTe, CdS, CdSe, CdTe, HgS, HgSe, or HgTe. In yet another aspect, the base or one or more legs are a semiconductor material that is one of: GaAs, InGaAs, InP, or InAs. In another aspect, MLN's can be prepared with a shell material that overcoats the one or more legs and the base. A shell overcoat can improve the photoluminescence quantum yields, enhance the tolerance or stability of the MLN's during process incorporation of the MLN's into various biological applications. In an aspect, an MLN can comprise more than one shell overcoating and each shell overcoat may comprise of different thicknesses greater than or equal to 0.001 nm and less than or equal to 999.99 nm.

The shell of an MLN can also contribute toward decreasing blinking associated with fluorescence of MLN's. Blinking can occur when an electron moving from a first charge state to a second charge state is lost in a surrounding environment or does not return to a first charge state due to being temporarily lost. Blinking can prove detrimental to biological labeling and detection of biological parameters, however shelling of a base and one or more leg material can decrease the onset of blinking and enhance photostability of the MLN. In an aspect, the shell overcoating the base and one or more legs are any one or more of a II-VI semiconductor material or a III-V semiconductor material. In another aspect, the shell is made of a semiconductor wherein the semiconductor is one of MgS, MgSe, MgTe, CaS, CaSe, CaTe, SrS, SrSe, SrTe, BaS, BaSe, BaTe, ZnS, ZnSe, ZnTe, CdS, CdSe, CdTe, HgS, HgSe, or HgTe. In yet another aspect, the shell is a semiconductor material that is one of GaAs, InGas, InP, or InAs, GaP, GaSb, InAs, InP, InSb, AlAs, AlP, AlSb. Furthermore, in an aspect, the shell itself can be overcoated with another shell made of a II-IV semiconductor material or a III-V semiconductor shell.

In some instances, MLN's may be comprised of many assortments of materials. For example, an MLN may comprise the base and one or more legs material as CdSe and the shell material as ZnS. Also, other suitable compound semiconductors for base and one or more legs material or shell materials include Group II-VI semiconducting compounds such as such as germanium or silicon may also be feasible under certain conditions. In other embodiments, the MLN base and one or more legs material or shell materials may comprise a dielectric material such as SiC or SiN. Some metals such as Fe, Ni, Cu, Ag, Au, Pd, Pt, or Co may also be used in embodiments of the invention. The MLNs may also be comprised of phosphorus, which is beneficial for many biological therapeutic applications due to phosporous's biocompatibility in vivo. In another aspect, an MLN comprised of phosphorous material has reduced toxicity due to the absence of cadmium, lead, or mercury.

In an aspect, MLN's can be comprised of either organic or inorganic shell materials. The MLN's can comprise “y” shell layers wherein y is an integer and each respective layer provides MLN photostability, reduced light scattering, increased image contrast, decreased blinking, enhanced MLN tolerance to surrounding environments, or decreased toxicity due to the decreased likelihood of cadmium or lead releasing into the surrounding environment. In an aspect, shelled MLN's are capable of emitting light within a narrow wavelength band thus permitting the simultaneous use of a plurality of MLN's each respectively comprising a unique spectral signature due to MLN variations in size, shape, form, features (e.g. number of legs, length of leg, width of leg) spectral signatures without wavelength overlap (or with a small amount of overlap).

In one embodiment, MLN's can take the shape of non-spherical nanoparticles or tetrapod shaped particles, wherein the particles comprise a base and one or more leg material of a Group 12, 13, 14, or 15 metal or metalloid and a shell material that is a Group 15 or 16 element. In an aspect, MLN's can comprise cadmium, sulfur, selenium, or tellurium. In an embodiment, an MLN can comprise an average leg length ranging from about 5 nm to 200 nm. In another embodiment, the leg width of the one or more legs can be less than the leg length of the one or more legs. In an embodiment, one or more leg widths range from about 2 nm to 10 nm.

There are various synthesis methods for making MLN's including but not limited to growing (by applying high temperatures to the base material) one or more legs from a base material where “n” in an integer. In another aspect, MLN's can be synthesized by attaching ‘n’ number of legs to a base material where “n” in an integer. The method of attaching includes but is not limited to conjugation, magnetics, and bonding including; but not limited to, covalent, ionic, or hydrogen bonding, Van der Waals forces, or mechanical bonding. In an aspect. MLN's can he synthesized fro use in various commercial applications including but not limited to biological applications by synthesizing the MLN's in aqueous medium to allow for use in bio-imaging, biosensors, and other biological applications where water solubility of the MLN's are required. Furthermore, the emission wavelength of the MLN's can be easily tuned from ˜400 to ˜900 nm by changing the size of the leg lengths, the leg widths, the number of legs, or length of the non-leg edge.

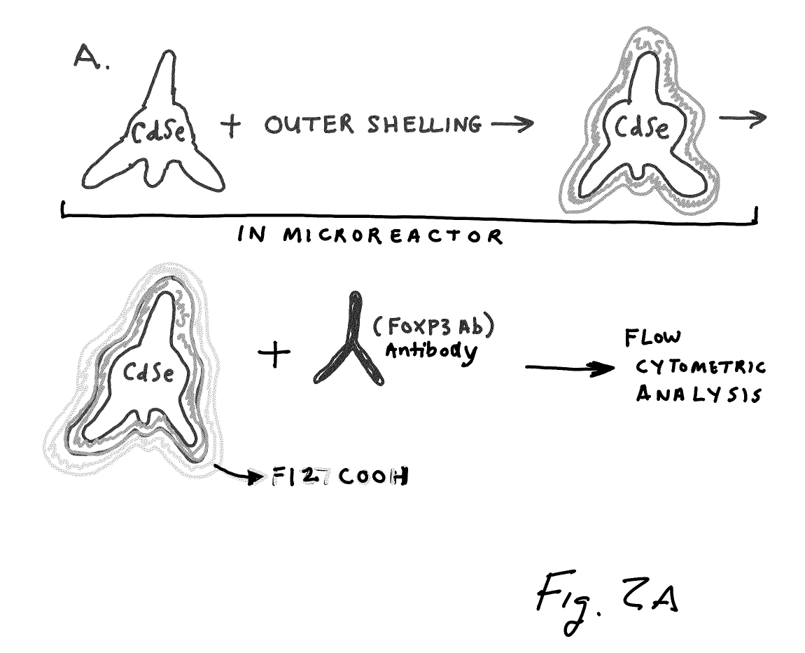

Turning now to FIG. 2A, shown is an MLN synthesized in a microreactor by a microreactor process. In an aspect, a micro-reactor can allow MLN synthesis reactions to be carried out in a continuous flow system. Further, the microreactor synthesis process can make use of kinetic parameters, reaction times, reagent ratio's, flow rates of reagent, and temperature adjustments to prepare MLN's in large-scale batches. In an aspect, syringes may be loaded with reagents and injected into the microreactor according to a controlled reaction time, reagent ratio, temperature control mechanism and specific reaction mixture. The microreactor process can scale-up the batch processes of making MLN's with both a base and one or more leg materials as well as shell material all within the microreactor. In some instances, the resulting MLN's are produced in kilogram quantities. Upon production a hydrophilic outer coating can be applied to the in order to be utilized in various biological detection applications.

In yet another aspect, the overcoating can comprise any one or more of a variety of biocompatible surface coatings such as ligand exchange, amphiphile encapsulation, and amphiphilic polymer coatings, as well as other coatings. Ligand exchange means overcoating the shell with bifunctional capping molecules (e.g. 1-thioglycolic acid, 1-thioglycerol, mercaptoethylamine, L-cysteine, 3-mercaptopropoinic acid, N-acetyle-L-cysteine, dihydrolipoic acid, . . . ). In another aspect, the shell can be overcoated with an amphiphile encapsulation, which occurs when a tetrapod article is encapsulated in an amphiphile (e.g. DSPE phospholipids, 1,2-distearoyl-sn-glyceroro-3-phosphoethanolamine-N-[methoxy[poly(ethylene glycol)]], DSPEmPEG 5000, 1,2-distearoyl-sn-glycero-3-phosphoethanolaminine-N-[amino(poly(ethylene glycol))2000], DSPE-PEG 2000 Amine, cyclodextrin). These coatings can transfer the MLN article into water (e.g. allow for soluble dispersion of the tetrapod article in aqueous solution).

In an aspect, the shell overcoating can be any one or more of: Pluronic F127, silicon, micelle, glass, polymeric oxide, oxide of phosphorous, or polymeric ligands, amphiphilic ligand, or hydrophilic thiol compound. With respect to some biological applications, the MLN article requires pairing to an associated biological molecule. To address this function the MLN articles need to be converted from their original hydrophobic exterior to an hydrophilic exterior.

In another aspect, MLN's can possess a pairing moiety connected to the shell coated one or more legs and base, and configured to connect to an targeting molecule. Pairing moieties allow MLN's to pair to targeting molecules such as antibodies, nucleic acids, polysaccharides, proteins, drugs, monoclonal antibodies, polyclonal antibodies, and other such targeting items. In an aspect, pairing moieties are chemical groups, or any combination of chemical groups, including, but not limited to, amino groups, carboxyl groups, azide groups, alkyne groups, hydrazine groups, aldehyde groups aminooxy groups, ketone groups, maleimide groups, thiol groups, or other such chemical groups.

Furthermore, an MLN can pair (via the pairing molecule alone or via a targeting molecule linked to the pairing molecule) to predefined targeted items through various mechanisms. In an aspect a predefined targeting item can be a biological material such as a cell, antigen receptor, biological marker, peptide, protein, or other such biological materials. The pairing moiety of an MLN can pair to targeting molecules such as antibodies, nucleic acids, polysaccharides, proteins, drugs, monoclonal antibodies, polyclonal antibodies, and other such targeting items. The targeting molecule can then find a complimentary bin logical marker, antigen, antibody, and so on for use in as a biological detection modality. Additionally, the MLNs may be conjugated to targeting molecules such as magnetic materials including but not limited to iron oxide particles, Fe/Pt, Co and Co/Fe, Fe, iron oxides, magnetic oxide, and other magnetic compositions for targeting purposes. The MLN with a magnetic property is useful for magnetic imaging and diagnostic imaging, and the magnetic attractive forces enhance targeting applications as well.

Turning now to FIG. 2B, shown is a flow cytometer analysis of a 590 mn MLN wherein n=4, thus the MLN is comprised of four legs protruding from the base. The MLN was synthesized using a micro-reactor continuous flow synthesis process. The MLN was bioconjugated to a FoxP3 antibody, whereby the MLN-FoxP3antibody was then exposed to T-regulatory cells from a sample of human blood. The flow cytometer indicates the presence of 590 nm wavelength emissions at specific intracellular sites where the correlation is drawn that the MLN-FoxP3antibody paired to a FoxP3 intracellular transcription factor. The FoxP3 intracellular transcription factor is present in T-regulatory cells thereby the detection of an MLN-FoxP3antibody-FoxP3intracellular transcription factor defines the presence of T-regulatory cells.

Furthermore, the flow cytometer analysis presents the number of cell counts that express FoxP3 intracellular transcription factor which is an indirect corollary of the quantity of T-regulatory cells present in the blood. Thus the number of flow cytometer events facilitates the identification of a number of T-regulatory cell counts (Number of cells that express FoxP3 intracellular transcription factor as determined by the detection of the 590 nm MLN) in a given blood sample. Additionally, the analysis suggests that MLN's synthesized by a microreactor continuous flow process are suitable materials for detection of biological materials in a biological sample.

Turning now to FIG. 3, the diagram shows the advantages of utilizing MLN's rather than spherical quantum dots. In an aspect, MLN's have several unique spectral signatures due to the adjustability of several MLN parameters including leg length, leg width, number of legs, base sizes, MLN shapes, MLN material compositions and other such adjustable features. However, a spherical quantum dot can only be tuned by adjusting the diameter of the sphere. Thus, MLN's have numerous more unique spectral signatures that can be obtained due to the great number of permutations and combinations of unique MLN's that can be created versus the limited range of different spherical quantum dots that can be obtained due to the variation of one feature only which is the spherical diameter

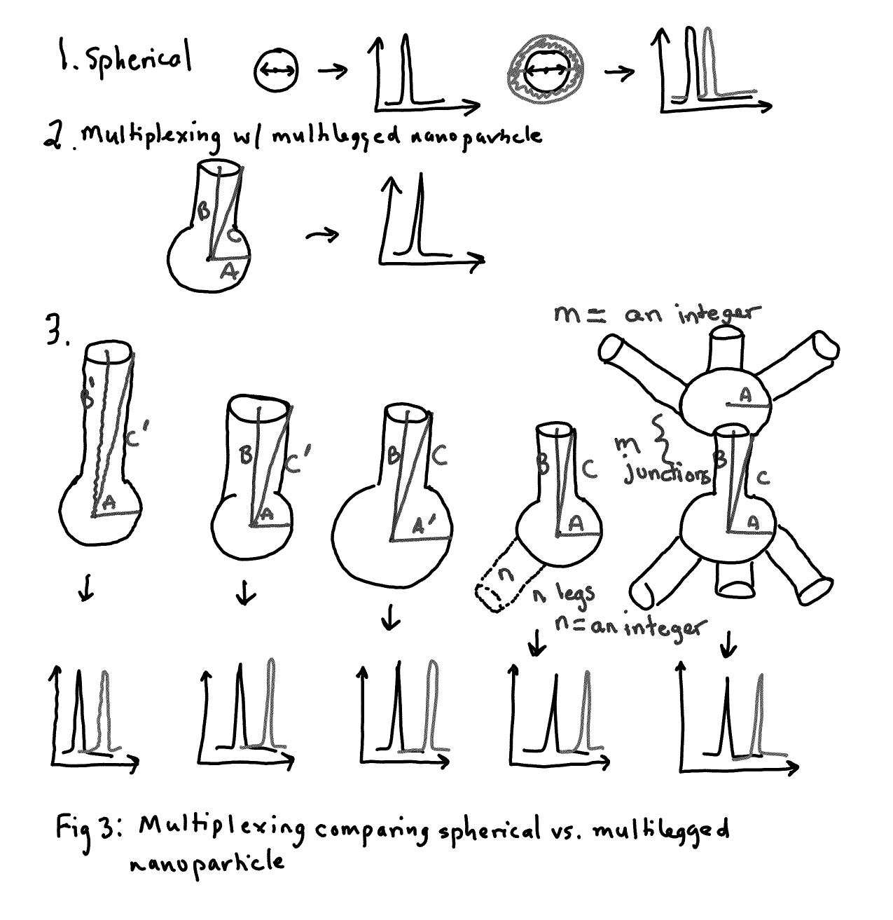

Turning now to FIG. 4, the diagram demonstrates the use of MLN's in a Polymerase Chain Reaction (PCR) fin molecular biology and pathogen detection. In an aspect, MLN's can improve the efficiency of PCR due to its size-tunable emission, broad excitation profile, narrow emission spectra, high photostability, and fluorescence at different wavelengths which lends itself to multiplexing. In an aspect, the PCR which integrates MLN's can be used for DNA cloning for purposes of sequencing, the diagnosis of hereditary diseases in a subject, genetic fingerprint identification, gene analysis, detection of infectious diseases, diagnosis of infectious diseases and other such applications. In such applications, the MLN's can improve PCR specificity, by adding different concentrations of unique MLN's to a PCR system. In an aspect, the MLN's are incorporated into PCR's as a multiplexing additive.

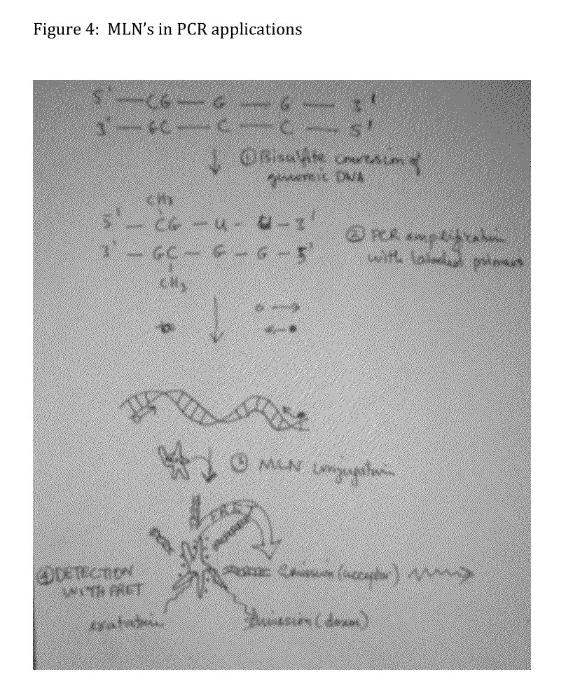

Turning now to FIG. 5, the diagram shows the use of MLN's in microarray technology such as DNA microarray's, RNA microarrays, protein microarray's, antibody microarrays, cellular microarrays, peptide microarrays, or tissue microarrays. A micromarray is a solid substrate such as a glass slide or thin-film cell that can be used to assay large amounts of biological materials in order to detect DNA, RNA, protein, or other such biological unit. An MLN can serve as a luminescent reporter in the microarray assay in order to detect numerous different biological units due to the MLN's vast multiplexing options.

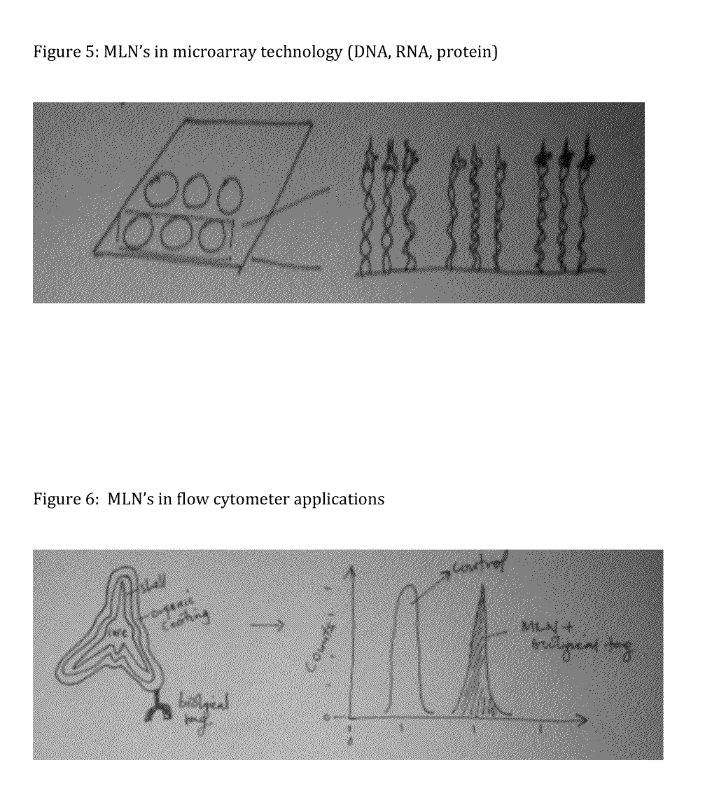

Turning now to FIG. 6, the diagram illustrates the use of MLN's in flow cytometer applications. A flow cytometer can be used to quantify properties (e.g. cell size, cell granularity) of single cells by labeling cell parameters with fluorescent probes. Flow cytometers take in a suspension of monodisperse cells and run them one at a time (single file) past a laser beam. As each cell passes through the laser beam, scattered and fluorescent light are quantitated. In an aspect, MLN's can be used to tag extracellular or intracellular parameters such as CD4+ or FoxP3. In an aspect, the MLN emit at greater intensities than current fluorescent probes and can rag a greater number of cellular parameters due to the numerous multiplexing options available. For instance, sixty MLN's, each with its own spectral signature (due to differences in adjustable MLN features such as number of legs), can label sixty different cellular parameters in order to allow for more specific identification and detection of cell types.

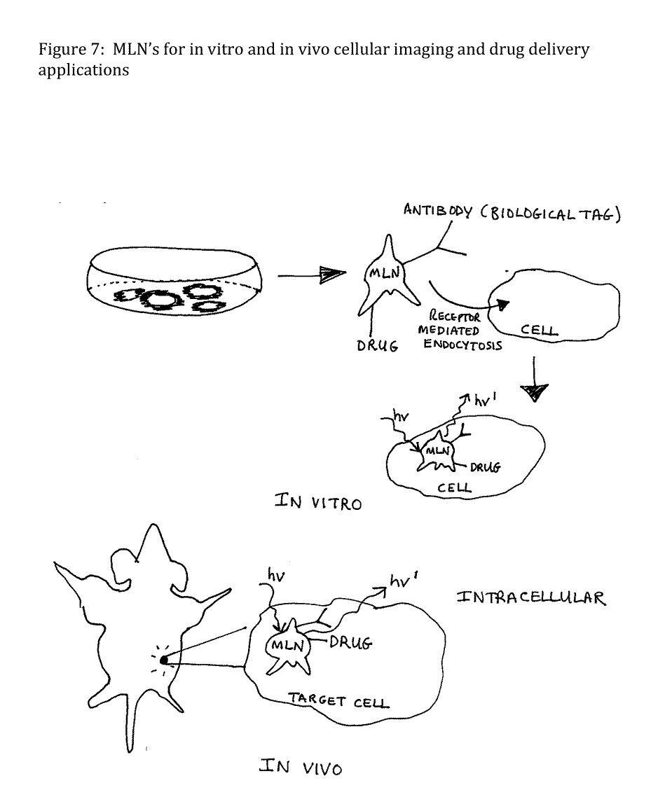

Turning now to FIG. 7, the diagram illustrates the use of MLN's for purposes of in vitro and in vivo cellular imaging or drug delivery applications. In an aspect, MLN's can be used in imaging applications, such as for reflectance fluorescent imaging, or tumor imaging in a subject. In another aspect, MLN's can be used in tumor targeting applications and drug delivery functionalities to deliver a drug payload (e.g. cancer therapy) to a targeted site (e.g. tumor cell) while simultaneously imaging the carrier particle. In an instance, an MLN can serve as a theranostic drug that both carries the drug payload to a targeted site and can be detected through imaging simultaneously. Furthermore, many drugs can be delivered to many sites, whereby each respective drug is bioconjugated to an MLN with a unique spectral signature in order to facilitate multiplexing identification of a treatment using a cocktail of different drugs.

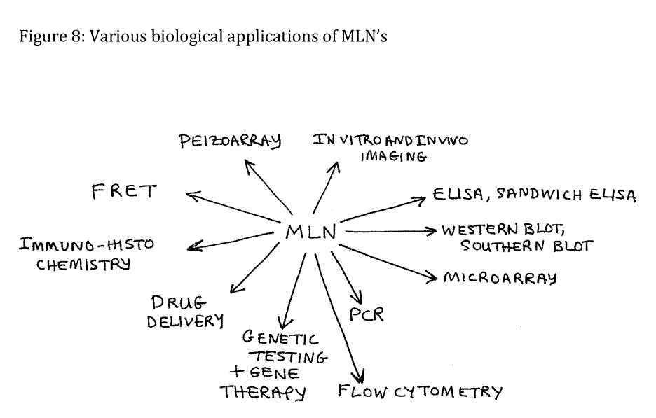

Turning now to FIG. 8, the diagram illustrates the advantages of using MLN's in various applications including, but not limited to Polymerase Chain Reaction technology applications, microarray technologies including but not limited to microarray analysis of DNA, RNA, or protein, flow cytometer application, in vitro application or in vivo application fir cellular imaging or drug delivery analysis, Western Blot application, Fluorescence Resonance Energy Transfer (FRET) analysis application, sandwich ELISA applications and other various biological applications

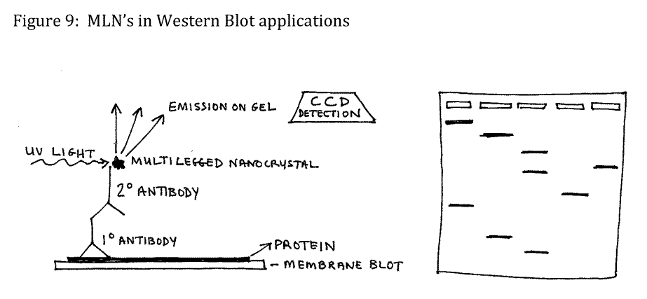

Turning now to FIG. 9, the diagram illustrates the incorporation of MLN's in Western Blot applications. Western blot, techniques are often used for protein immunodetection. In an aspect, MLN's can be used to simultaneously detect numerous protein types simultaneously whereby such multiplex detection of proteins offer the measurement of more proteins than can be identified via use of spherical quantum dots. Furthermore, MLN's possess greater tenability than spherical quantum dots due to the greater number of adjustable features which result in a greater number of unique spectral signatures associated with each MLN.

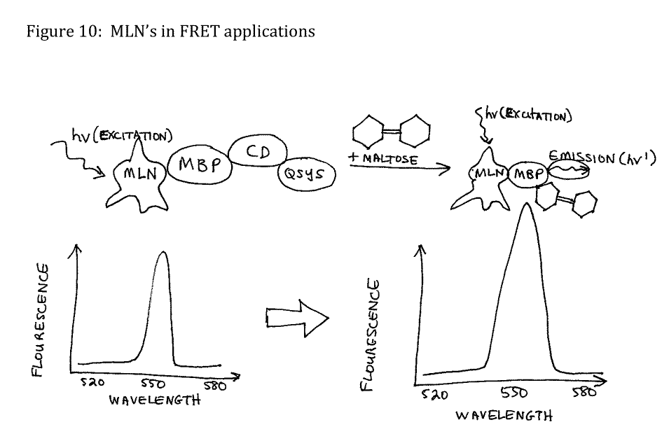

Turning now to FIG. 10, the diagram illustrates the incorporation of MLN's in FRET applications. In an aspect, the MLN's can be advantageous in FRET analysis in its ability to emit unique spectral signatures, possess long durations of stability, reduction of quenching from intra-molecular energy transfers, resistance to photobleaching, and for purposes of all disclosed applications is cheap to produce due to the large-scale microreactor synthesis process. Additionally, MLN's can add a significant advantage to FRET applications due to its multiplexing capabilities.

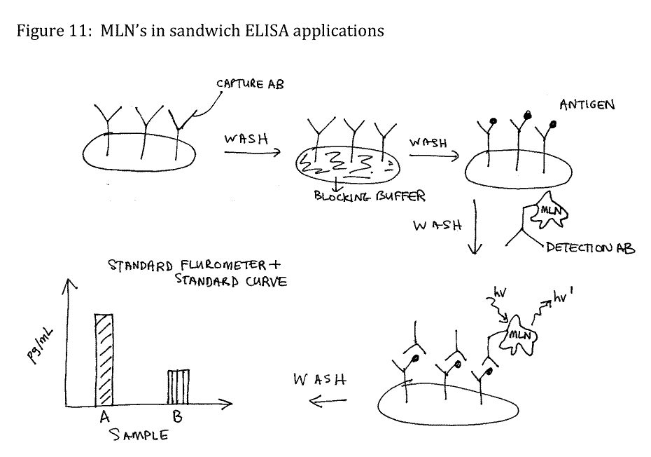

Turning now to FIG. 11, the diagram illustrates the incorporation of MLN's in sandwich ELISA applications. A sandwich ELISA measures the amount of antigen between two layers of antibodies; a capture antibody and a detection antibody. In an aspect, a monoclonal antibody (e.g. usually the detection antibody that can quantify small differences in antigen) or polyclonal antibody (e.g. usually the capture antibody that anchors down as much antigen as possible) can be used as the capture and detection antibodies. In an aspect, MLN's can be used to detect multiple antigens through several MLN's conjugation to several respective antibodies, each different MLN possessing its own spectral signature, thus the MLN can facilitate multiplexed identification of several antigens in each ELISA.

### Various Use's and Features of MLN's

Multiplexing Assays. Multiplexing is the simultaneous amplification, labeling and detection of many different targets with one single excitation source. In an aspect, MLN's can allow for the tracking of several parameters given the adjustable features that give each MLN distinguishing, characteristics such as a unique spectral signature or emission of a unique wavelength. In one embodiment, each MLN of a different spectral signature is paired to a unique antibody, thus for each complimentary antigen an antibody pairs to, the antibody-antigen pairing will emit a unique spectral signature subsequent to excitation of each unique MLN paired to the antibody-antigen. For instance, an MLN emitting at a 530 mn wavelength is paired to a FoxP3 antibody, an MLN emitting at a 630 nm is paired to a CD4 antibody, and an MLN emitting at a 730 nm wavelength is paired to a CD25 antibody. Upon the MLN-FoxP3antibody-FoxP3antigen, MLN-CD4antibody-CD4antigen, and MLN-CD25antibody-CD25antigen respective pairings, the excitation of such respective pairs will present three unique spectral signatures to identify each of FoxP3 antigen, CD4 antigen and CD25 antigen simultaneously.

Furthermore, in an aspect, MLN's are capable of identifying hundreds of analytes in a sample simultaneously. In an aspect, multiple analytes can be identified concurrently via use of MLNs, each of which may emit at various wavelengths due to varying leg lengths, leg widths, base lengths, number of legs, base edge lengths, or material compositions all of which are excitable by one single excitation source. In one embodiment, the an MLN exhibiting a unique spectral signature is paired to a unique antibody in order to emit such unique spectral signature as a detectable wavelength. If one antibody for instance has five different MLNs bound to its surface, each MLNs emitting at a different wavelength, then an instrument can detect the nature of such target, by reading the five wavelengths emitted from such target in sequence. Likewise, sequence variations of MLNs with different wavelengths attached to targets can result in hundreds of parameters being tagged in a single instance.

Additionally, in an aspect, MLN's can be tuned to emit light to nearly any color in the visible spectrum, with very high quantum yields and at very narrow spectral distributions (10 to 40 nm half width maximums). MLN's serve an important biological advantage in that the half maximum width of MLNs can be smaller than the conventional spherical quantum dots the benefits of this is that it's easier to decipher more parameters when conducting real time analysis or multiplexing due to more emission spectrums that can fit for a defined range of detection wavelengths.

### Complexation of MLN's in Various Other Applications

In an aspect, assay kits, and other enhanced biological detection tools utilizing MLN's can achieve increased sensitivity, require less sample volumes, and measure multiple antigens in real time. Existing diagnostic techniques in the biological sciences are enhanced by these properties including but not limited to: flow cytometer applications, IHC, Western Blot, ELISA, PCR arrays, peizoarrays, polymerase chain reaction technology applications, microarray technology, in vitro cellular imaging application, in vivo cellular imaging application, in vitro drug delivery application, in vivo drug delivery application, FRET analysis application, sandwich ELISA application, genetic testing application, or other such biological application. In an aspect, such applications can be made by pairing each MLN to a targeting molecule such as: an antibody, a nucleic acid, a protein, a polysaccharide, a small molecule, avidin, streptavidin, a biotin, a antidigoxiginen, a monoclonal antibody, a polyclonal antibody, a nucleic acid, monomeric nucleic acid, oligomeric nucleic acid, a protein, a polysaccharide, a sugar, a peptide, a drug, carbohydrate, ligands.

In an aspect, MLN's can be used for genetic testing whereby MLNs are paired to genetic material. The MLN's can be positively charged via pairing to NH4+ following shelling and become capable of binding to negatively stranded nucleic acid through electrostatic interactions. Furthermore, the electrostatic interactions allow for selective gene detection of RNA, DNA, and plasmids sometimes simultanously. In an aspect, more genetic parameters can be measured per single real time assay by changing adjusting the features of MLNs to produce several MLN's each emitting at a unique spectral signature upon excitation with UV or blue light. Furthermore, pairing MLN's to genetic material can achieve selective gene delivery of RNA, DNA, and plasmids to specific target cells both in vitro and in vivo. Unlike spherical quantum dots, the MLNs configuration allows for the pairing of more genetic material per singular unit of nanoparticle due to the greater surface area for pairing (e.g. can pair genetic material to numerous legs, the base, etc.).

The MLN can be further grown as inorganic dendrimers called BMLN's whereby the inorganic BMLNs are used as a method to increase both transfection efficiency and pharmokinetics. In an aspect, a branched multi-leg luminescent nanoparticle compound is comprised of at least two bases, each of the at least two bases respectively having at least one leg protruding there from, wherein the at least two bases or the respective at least one legs are coupled, and wherein the at least two bases and the at least one legs comprise a luminescent semiconductor nanoparticle; a shell that coats the at least two bases and the at least one legs; and a pairing moiety connected to the shell coated the at least two bases and the at least one legs and configured to connect to an targeting molecule.

### Miscellaneous Aspects of MLN's

Overview of Property enhancements: MLNs can also be complexed to drugs. The MLN's can be modified to have a unique shelling and associated surface chemistry so it can be bound to any drug of choice given the appropriate structure of the drug. In an aspect, the unique structure of MLN's allow for specific property enhancements as compared to spherical quantum dots such as:

Decreased Blinking: There is less blinking based on the continuous flow manufacturing process. Furthermore, by providing additional shell layers to the MLN's using an inorganic shell, polymer coating, or both an inorganic shell with a polymer overcoating, the net effect prevents the core from breaking down which results in less blinking and enhances photoluminescence as well as photostability.

Decreased In vivo and In vitro cellular toxicity: MLNs that are shelled present less cellular toxicity. By shelling the core there is less degradation of the core into the surrounding cellular Matrix preventing intracellular toxicity.

Increased cellular uptake: For each nanoparticle of a MLNs versus a spherical nanocrystal, the shape properties allow for greater cellular uptake because most cells in the living system use polyanionic receptors as a method of conducting either receptor mediated endocytosis or phagocytosis. By affecting the overall spatial charge distribution around the nanomaterials the MLN's enhance cellular uptake. Furthermore, the multi-leg configuration allows the MLN's to act as building blocks for BMLN's that will allow for greater transfection of drug per unit of nanoparticle, leading to a more favorable pharmacokinetic profile and therapeutic index to effectively maximize therapeutic efficacy of the drug while minimizing systemic and regional side effects.

Selective Targeting: The ability to modify the surface chemistry of the MLN's allows for selective targeting of specific cell types including but not limited to cancer cells, neurons, and other cells within a subject. In an instance, tissue factor receptors can be paired to on the surface of MLN's in order to enhance uptake of the tissue factor receptors by pancreatic cancer cells. Another advantage of MLN's are its theranostic application whereby the composition of a single MIN unit can be changed whereby the composition of the legs are comprised of different materials than the base, and such material variations can add to the production of complex inorganic dendrimers such as different BMLN's with unique properties. For instance, in an aspect, the legs of an MLN can comprise magnetic properties (e.g. made from iron oxide material) and the base can be a bioluminescent semiconductor to allow for magnetic and luminescent drug delivery MLN's.

Excitation of MLN's: In an aspect, MLNs are capable of being excited by electromagnetic radiation over a broad range of wavelength's from x-ray to ultraviolet to visible light to infrared light. Also, in an aspect, the MLNs are capable of excitation from bombardment with a particle beam such as an electron beam.

What is claimed is:

1. A multi-leg luminescent nanoparticle, comprising:
one or more legs extending from a base wherein the one or more legs and the base comprise a luminescent semiconductor nanoparticle;
a shell that coats the one or more legs and the base; and
a pairing moiety connected to the shell coated one or more legs and base, and configured to connect to an targeting molecule.

2. The nanoparticle of claim 1, wherein the base and one or more legs are a semiconductor or a III-V semiconductor.

3. The nanoparticle of claim 2, wherein the base and one or more legs are a II-VI semiconductor.

4. The nanoparticle of claim 2, wherein the base and one or more legs are a semiconductor.

5. The nanoparticle of claim 3, wherein the semiconductor is MgS, MgSe, MgTe, CaS, CaSe, CaTe, SrS, SrSe, SrTe, BaS, BaSe, BaTe, ZnS, ZnSe, ZnTe, CdS, CdSe, CdTe, HgS, HgSe, or HgTe.

6. The nanoparticle of claim 4, wherein the semiconductor is GaAs, InGaAs, InP, or InAs.

7. The nanoparticle of claim 1, wherein the shell semiconductor is a II-VI semiconductor or a III-V semiconductor.

8. The nanoparticle of claim 7, wherein said shell is a II-VI semiconductor or a III-V semiconductor.

9. The nanoparticle of claim 8, wherein the semiconductor is MgS, MgSe, MgTe, CaS, CaSe, CaTe, SrS, SrSe, SrTe, BaS, BaSe, BaTe, ZnS, ZnSe, ZnTe, CdS, CdSe, CdTe, HgS, HgSe, or HgTe.

10. The nanoparticle of claim 9, wherein the semiconductor is GaAs, InGaAs, InP, or InAs.

11. The nanoparticle of claim 1, wherein the base and one or more legs is CdSe and the shell is ZnS.

12. The nanoparticle of claim 1, wherein the multi-legged luminescent nanoparticle further comprises a glass coating on the shell.

13. The nanoparticle of claim 12, wherein the glass coating comprises a polymeric oxide.

14. The nanoparticle of claim 1, wherein the pairing moiety comprises any of a thiol moiety, F127COOH, alkyl group, propyl group, N-(3-aminopropyl)-3-mercapto-benzamide, 3-aminopropyl-trimethoxysilane, 3-mercaptopropyltrimethoxysilane, 3-maleimidopropyl-trimethoxysilane or 3-hydrazidopropyl-trimethoxysilane, diacetylenes, acrylates, acrylamides, dihydrolipoic acid, trioctyl phosphine, vinyl, and styryl.

15. The nanoparticle of claim 1, wherein the targeting molecule is chosen from an antibody, a nucleic acid, a protein, a polysaccharide, a small molecule, avidin, streptavidin, a biotin, a antidigoxiginen, a monoclonal antibody, a polyclonal antibody, a nucleic acid, monomeric nucleic acid, oligomeric nucleic acid, a protein, a polysaccharide, a sugar, a peptide, a drug, carbohydrate, ligands.

16. The nanoparticle of claim 1, wherein the shell is water-soluble.

17. The nanoparticle of claim 1, wherein the shell has any number of shell coated layers.

18. The nanoparticle of claim 1, wherein the nanoparticle is used in any one or more of a polymerase chain reaction technology application, microarray technology, flow cytometer application in vitro cellular imaging application, in vivo cellular imaging application, in vitro drug delivery application, in vivo drug delivery application, western blot application, FRET analysis application, sandwich ELISA application, or genetic testing application.

19. A branched multi-leg luminescent nanoparticle compound, comprising:
at least two bases, each of the at least two bases respectively having at least one leg protruding there from, wherein the at least two bases or the respective at least one legs are coupled, and wherein the at least two bases and the at least one legs comprise a luminescent semiconductor nanoparticle;
a shell that coats the at least two bases and the at least one legs; and
a pairing moiety connected to the shell coated the at least two bases and the at least one legs and configured to connect to an targeting molecule.

20. The multi-leg luminescent nanoparticle of claim 1 synthesized by a microreactor process.
